(19) United States
(12) Patent Application Publication
(10) Pub. No.: US 2019/0072493 A1
(43) Pub. Date: Mar. 7, 2019

(54) DEVICE AND METHOD FOR ON-CHIP CHEMICAL SEPARATION AND DETECTION

(71) Applicants: Alan X. Wang, Corvallis, OR (US); Xianming Kong, Fushun (CN); Kenneth Squire, Corvallis, OR (US)

(72) Inventors: Alan X. Wang, Corvallis, OR (US); Xianming Kong, Fushun (CN); Kenneth Squire, Corvallis, OR (US)

(73) Assignee: Oregon State University, Corvallis, OR (US)

(21) Appl. No.: 16/120,904

(22) Filed: Sep. 4, 2018

Related U.S. Application Data
(60) Provisional application No. 62/554,309, filed on Sep. 5, 2017.

(51) Int. Cl. G01N 21/65; G01N 21/552; G01J 3/44

(52) U.S. Cl. CPC G01N 21/658; B82Y 40/00; G01J 3/44; G01N 21/554

(57) ABSTRACT

Microfluidic diatomaceous earth analytical devices (μDADs) comprising highly porous photonic crystal biosilica channels are disclosed. The μDADs can simultaneously perform on-chip chromatography to separate small molecules from complex samples and acquire the surface-enhanced Raman scattering spectra of the target chemicals with high specificity. The ultra-small dimensions of the diatomaceous earth microfluidic channels and the photonic crystal effect from the fossilized diatom frustules allow unprecedented sensitivity down to ppb-level.

### CROSS REFERENCE TO RELATED APPLICATION

This application claims the benefit of the earlier filing date of U.S. provisional patent application No. 62/554,309, filed Sep. 5, 2017, which is incorporated herein by reference in its entirety.

### ACKNOWLEDGMENT OF GOVERNMENT SUPPORT

This invention was made with government support under Grant No. EB018893 awarded by the National Institutes of Health. The government has certain rights in the invention.

### FIELD

The present invention concerns sensors, particularly sensors that combine aspects of chromatography (TLC) and surface-enhanced Raman spectroscopy (SERS).

### BACKGROUND

Recently, there has been an increasing demand for accurate and instant on-site identification of a variety of chemical and biological targets, including ultra-sensitive detection of toxins and contaminants for food and environmental safety, disease biomarkers for health care, drug markers for health care and law enforcement, and explosives and gunpowder residues for homeland security. Current detection platforms for sensitive detection of analytes in trace amounts include high performance liquid chromatography (HPLC) and gas chromatography in tandem with mass spectrometry (GC-MS). Although these methods are accurate and reliable, they are expensive, time-consuming, insufficiently portable and require skilled personnel for operation.

Surface-enhanced Raman scattering (SERS) spectroscopy has attained considerable interest as a sensitive detection method. Raman signals of a single molecule have been observed on the surface of metallic nanostructures with enhancement factors (EF) as high as 1014. These results establish the ultra-high sensitivity of SERS. Another exclusive advantage of SERS is the wealth of inherent information that can be ascertained about the chemical and molecular composition of a sample. This capability makes SERS a powerful and nondestructive sensing technique widely used, for instance, in chemical analysis, hazardous material detection, forensic justification, and art identification.

In reality, test samples are often complex biological or chemical samples and/or contain two or more constituents, which makes detecting individual constituents from mixed samples very challenging for SERS analysis. One primary reason for this difficulty is that the affinity between molecules and metal surfaces varies significantly during the SERS measurement process. Only those dominant molecules on the metal surface can be detected. For instance, it is extremely difficult to directly detect small molecules using SERS from biofluid samples or other samples having high salt concentrations because the salt strongly influences the stability of both metallic colloids used for SERS and the biomolecules. Furthermore, the complicated composition of a biofluid sample precludes accurate SERS detection of individual target molecules. Accordingly, appropriate separation techniques are needed before accurate SERS measurements can be obtained.

Microfluidic paper-based analytical devices (μPADs) have fostered a new spectrum of microfluidic devices for point-of-care diagnosis and biosensing. μPADs can be fabricated by simple, low-cost processes using conventional photo- or soft-lithographic techniques, utilizing either photoresists or wax printing. μPADs provide a number of advantages, including: 1) availability of ubiquitous and extremely cheap cellulosic materials; 2) capillary flow, which enables fluid transport without using any external pump; and 3) compatibility with many chemical and biomedical applications. Many different chemical and biological assays have been performed using μPADs, including detection of glucose, protein (albumin), cholesterol, lactate, alcohol, enzymes (transaminase12 and galactosidase13), and heavy metals. μPADs also have been used as a platform for ELISA. And inkjet-printed paper SERS substrates have been used for chromatographic separation and detection of target analytes from complex samples, which opened a new route for on-chip chemical sensing.

Other than μPADs, porous silica materials and devices also have attracted considerable attention for biosensing due to their large surface area and pore volume that contribute to achieving high sensitivity. The high porosity of these materials facilitates immobilization of target molecules not only on the external surface of the substrate but also inside the pores. This enables large amounts of sensing molecules to be loaded on the high porosity materials, which facilitates instant responses and high sensitivity. The optical transparency, on the other hand, permits optical detection through the bulk of the material. The surface groups and biocompatibility of these porous silica materials also contribute to making porous silica a potentially useful material for biosensing. For example, polymer and colloidal silica porous composites have been fabricated for nucleic acid biosensing. These composites were synthesized and used as enzyme immobilization carriers to fabricate glucose biosensors. And synthesized SiO₂ materials have been used as enzyme immobilization carriers to fabricate glucose biosensors. However, pores in sol-gel derived silica lack a high degree of order, which results in nonlinear diffusion paths, and consequently slow analyte diffusion to the sensing molecules. Some fraction of the sensing molecules might even be unreachable leading to low response.

Diatoms are unicellular, photosynthetic biomineralization marine organisms that have a biosilica shell that is referred to as a frustule. The two dimensional (2-D), highly periodic diatom surface pores provide unique optical, physical, and chemical properties. Photoluminescence-based diatom biosensors have been developed for TNT sensing. And a highly-selective biosensor for immunocomplex detection has been developed by modifying diatom frustules (Coscinodiscus concinnus) with antibodies. The present inventors have previously developed an in-situ growth method for depositing silver (Ag) nanoparticles (NPs) on diatoms to allow ultrasensitive TNT sensing.

Diatomaceous earth comprises fossilized remains of ancient diatoms and is a type of naturally abundant photonic crystal biosilica having high porosity. Diatomaceous earth has similar properties to diatoms, such as a highly porous structure, excellent adsorption capacity, and photonic crystal effects. And there are billions of tons of fossilized diatoms on earth.

### SUMMARY

The present disclosure describes embodiments of a device that can be used for a number of purposes, such as label-free sensing of small molecules from complex biological samples by on-chip chromatography and surface-enhanced Raman scattering (SERS). One embodiment of the device comprised a microfluidic diatomaceous earth analytical device (μDAD), comprising a multi-scale, hierarchically porous photonic crystal biosilica channels, e.g a sensor fabricated on a diatomaceous earth porous microchannel chip. This device can be used to separate numerous compounds in complex samples for rapid detection with high specificity and extremely high sensitivity. SERS detection of small molecules on the diatomaceous earth surface of the microchannels can be further enhanced by metal nanoparticle deposition.

A system also is disclosed. Certain embodiments of the system comprise a separation and SERS analysis device comprising at least one microchannel comprising or formed from diatomaceous earth. The analysis device is used in combination with a Raman spectrometer.

Certain disclosed embodiments concern a method comprising separating a composition comprising at least two separate components into a first component and a second component using diatomaceous earth as a stationary chromatography phase, wherein at least one of the first and second components can be detected and identified by Raman spectroscopy. The separated components are then analyzed by Raman spectroscopy. The device can be used to detect a variety of molecules, including drug molecules, gunpowder and explosives residues, polycyclic aromatic hydrocarbons (PAHs), metabolic by-products, pesticides and antibiotics, and other organic molecules of interest. The specific examples presented herein are not intended to limit the scope of the disclosure and a person of ordinary skill in the art will recognize that the present invention will be useful for detecting any molecule with a Raman spectra. Metal nanoparticles may be applied to the first and second components after separation to increase Raman signal intensity. The metal nanoparticles typically are selected from gold (Au) nanoparticles (NPs), silver (Ag) nanoparticles, copper (Cu) nanoparticles, aluminum (Al) nanoparticles, or combinations thereof. Au and/or Ag nanoparticles, particularly Au and/or Ag nanoparticles having a diameter of from about 50 nanometers to about 60 nanometers, generally have been used for disclosed features of the present invention.

Devices according to the present disclosure are made by depositing diatomaceous earth, or processed or modified materials made from diatomaceous earth, all of which materials are generally referred to herein as diatomaceous earth, on to a substrate, such as a glass slide, plate or cellulosic substrate. For certain disclosed embodiments, the diatomaceous earth is applied by spin coating dried diatomaceous earth onto the substrate. The dried diatomaceous earth may be added to an appropriate dispersing fluid, such as carboxymethyl cellulose, to form a diatomaceous earth dispersion prior to applying the diatomaceous earth dispersion to the substrate. A desired layer thickness is deposited, such as from greater than 0 μm to at least 100 μm, preferably greater than zero to about 50 μm, greater than 5 μm to about 50 μm, and typically from about 15 μm to about 20 μm. The deposited material may be deposited as, or formed into, a microchannel comprising or made from diatomaceous earth. The microchannel may be fluidly associated with an eluent reservoir.

Certain disclosed embodiments particularly concern analyzing biofluids by performing a chromatographic separation of a biological sample using a diatomaceous earth stationary phase. SERS analysis is then performed on components separated from the biological sample.

The present invention provides a substantial improvement over prior known technologies. For example, certain disclosed embodiments provide a substantial SERS intensity increase, in some embodiments an increase of up to 70×, at least a 10× increase in the level of detection, and unprecedented ppb sensitivity relative to technologies using a non-diatomaceous-earth-based stationary phase material.

The foregoing and other objects, features, and advantages of the disclosure will become more apparent from the following detailed description, which proceeds with reference to the accompanying figures.

### BRIEF DESCRIPTION OF THE DRAWINGS

FIG. 1 is a schematic representation of a TLC-SERS detection method for detecting a target molecule from a mixture comprising at least one SERS detectable analyte using porous diatomaceous earth biosilica as a stationary phase

FIGS. 2A-2D provide a schematic diagram illustrating components of exemplary TLC-SERS technology and method for using the technology:

FIG. 2A illustrates using a commercial hand-held Raman spectrometer for on-site, on-chip analyte detection;

FIG. 2B is optical image of a single diatom in diatomaceous earth illustrating photonic crystal effects;

FIG. 2C illustrates TLC-SERS arrayed channels for high throughput universal sensing; and

FIG. 2D illustrates certain methods step used to conduct TLC-SERS sensing for certain disclosed exemplary embodiments.

FIG. 3 is an SEM image of Au colloids as prepared in accordance with disclosed exemplary embodiments.

FIG. 4 is a UV-Vis absorption spectrum (absorbance versus wavelength) of Au colloids prepared in accordance with disclosed exemplary embodiments.

FIG. 5 provides Raman spectra of a diatomaceous earth plate both with and without applied Au nanoparticles, establishing that Au nanoparticles substantially increased the intensity of the Raman signal.

FIG. 6 provides superimposed Raman spectra of 4-mercaptobenzoic acid (MBA, 10 ppm) with and without applied Au nanoparticles, establishing that Au nanoparticles substantially increased the intensity of the Raman signal.

FIG. 7A provides superimposed Raman spectra of 100 ppm of mercaptobenzoic acid on a diatomaceous earth chromatography plate with six different concentrations of gold nanoparticles applied thereto, illustrating a direct, positive correlation between increased Raman signal intensity and increased gold nanoparticles concentration.

FIG. 7B is a graph of concentration factor versus Raman signal intensity establishing that Raman signal increase occurs up to 100× an initial concentration of gold nanoparticles, whereas at higher concentrations of gold nanoparticles the Raman signal intensity decreases.

FIG. 8 is an SEM image of diatomaceous earth with honeycomb structure (FIGS. 8A and 8B) and a cross sectional microscopy image (FIG. 8C) of a TLC plate comprising diatomaceous earth applied by spin coating.

FIG. 9A is an SEM image of a diatomaceous earth porous microchannel.

FIG. 9B is a honeycomb-like diatomaceous earth.

FIG. 9C is an optical image of a porous microchannel after 100 ppm pyrene migration and illumination using UV light.

FIGS. 10A-10D is an image of a single diatom under the microscope (FIG. 10A-FIG. 10D=far to near), showing the diffraction pattern from photonic crystal.

FIG. 11A provides SERS spectra of pure Raman probe molecules mercaptobenzoic acid (MBA), R6G and Nile Blue (NB), as well as pyrene.

FIG. 11B provides Raman spectra of various mixtures of these components.

FIGS. 12A-12D provide digital images of TLC plates used to separate mixtures (mixture 1—pyrene and MBA; mixture 2—pyrene and R6G; mixture 3—pyrene and nile blue) separated by diatomaceous earth (FIGS. 12A and 12C) and silica gel (FIGS. 12B and 12D) into mixture components, where the spots after separation were visualized with UV light (FIGS. 12A and 12B) and iodine colorimetry (FIGS. 12C and 12D).

FIG. 13A provides SERS spectra of individual mixture components after separating a mixture of pyrene and MBA into the components using a diatomaceous earth TLC plate.

FIG. 13B provides SERS spectra of individual mixture components after separating a mixture of pyrene and R6G into the components using a diatomaceous earth TLC plate.

FIG. 13C provides SERS spectra of individual mixture components after separating a mixture of pyrene and Nile blue into the components using a diatomaceous earth TLC plate.

FIGS. 14A and 14B provide SERS spectra of a mixture of pyrene and MBA at different concentrations separated by diatomaceous earth TLC plates.

FIGS. 14C and 14D provide SERS spectra of a mixture of pyrene and MBA at different concentrations separated by silica gel TLC plates.

FIG. 15A is a Raman mapping image of MBA (10 ppm) on diatomaceous earth TLC plates.

FIG. 15B is a Raman mapping image of MBA (10 ppm) on silica gel TLC plates.

FIG. 16A provides SERS spectra of plasma with different concentrations of phenethylamine (PEA) separated by diatomaceous earth.

FIG. 16B provides SERS spectra of plasma with PEA with DNA added and separated by diatomaceous earth.

FIG. 17 provides digital images of different concentrations of pyrene separated by a diatomaceous earth porous microchannel and by a normal diatomaceous earth chromatography plate, followed by visualization with UV light.

FIG. 18A provides fluorescence spectra of the different concentrations of pyrene separated by a normal diatomaceous earth chromatography plate.

FIG. 18B provides fluorescence spectra of the different concentrations of pyrene separated by a diatomaceous earth porous microchannel.

FIG. 19 provides SERS spectra of human plasma with different concentrations of cocaine separated by diatomaceous earth porous microchannel chip.

FIG. 20A provides SERS spectra of pure substance of MBA, pyrene and the corresponding mixture.

FIG. 20B provides SERS spectra of different spots on diatomaceous earth porous microchannel after chromatography separation.

FIG. 21A provides SERS spectra of the first spot from a pyrene and MBA 1/1 mixture at different concentrations separated by a diatomaceous earth porous microchannel chip.

FIG. 21B provides SERS spectra of the second spot from the pyrene and MBA 1/1 mixture from FIG. 21A at different concentrations separated by a diatomaceous earth porous microchannel chip.

### DETAILED DESCRIPTION

### I. Definitions

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. All references, including patents and patent applications cited herein, are incorporated by reference.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is expressly recited.

Unless explained otherwise, all technical and scientific terms used herein have the same meaning as commonly understood to one of ordinary skill in the art to which this disclosure pertains. Although methods and materials similar or equivalent to those described herein can be used in the practice or testing of the present disclosure, suitable methods and materials are described below. The materials, methods, and examples are illustrative only and not intended to be limiting.

The terms “diatomite” and “diatomaceous earth” as used in U.S. provisional application No. 62/554,309 refer to a powder formed from sedimentary rock that mainly comprises skeletal remains of diatoms. In the present application, the powder is referred to as diatomaceous earth. Diatomaceous earth is sold by Sigma-Aldrich sells as Celite®.

Micro as used herein, such as in reference to a microchannel, refers to having at least one dimension, such as width and/or depth, that is one millimeter (1000 microns) or less.

### II. Introduction

Since the initial TLC-SERS work, TLC-SERS has been successfully applied to the separation and identification of various analytes from mixtures, including on-site detection of organic pollutants in environmental water samples, identification of the ingredients in medicinal herbs, identifying foodborne contaminants, and monitoring chemical reactions. TLC performance mainly depends on the chromatographic materials and eluents. Continuous improvements in stationary TLC phases have enabled shorter separation times, higher resolution and greater sensitivity. Recent developments in stationary TLC phases include silicon-carbon, multiple-component materials having an aporous structure, electro-spun nano-fibers doped with a photoluminescence indicator, and nanostructured thin films produced via glancing angle deposition.

TLC plates are commercially available for most TLC-SERS methods, and silica gel is the most widely used stationary phase material. However, silver nanorod arrays have also been used as the stationary phase for TLC-SERS. The nanorod arrays were prepared by oblique angle deposition (OAD). And inkjet-printed paper substrates were used for TLC-SERS to detect melamine in food products. However, these TLC substrates were not optimized to enhance the hot-spots for SERS sensing and the limit of detection (LOD) for these existing TLC-SERS technologies is not sufficient for many applications.

Diatomaceous earth comprises fossilized remains of diatoms, a type of hard-shelled algae. Diatomaceous earth is a natural photonic bio-silica from geological deposits. It has a variety of unique properties including a highly porous structure, excellent adsorption capacity, and low cost. In addition, the two dimensional (2-D) periodic pores on diatom frustules enable hierarchical nanoscale photonic crystal features. Metallic nanoparticles (NPs) located near or inside periodic nanopores of diatoms can form hybrid photonic-plasmonic modes via theoretical analysis and experimental results. These photonic-plasmonic modes further increase the local electric field near the plasmonic substrate and additional SERS enhancement was achieved.

A. Devices and Systems

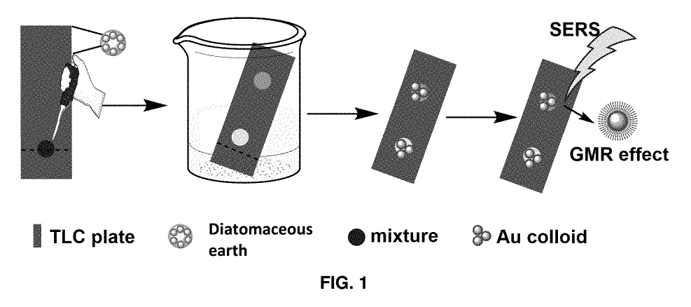

FIG. 1 is a schematic representation of one embodiment of the present invention. Certain disclosed embodiments concern using TLC plates comprising diatomaceous earth as a stationary phase to separate target molecules from mixtures. The diatomaceous earth also functions as photonic crystals to enhance the SERS sensitivity.

With specific reference to FIG. 1, a TLC plate is made using diatomaceous earth as a stationary phase. A sample comprising two separable components is spotted at a bottom portion of the TLC plate. The TLC plate is then placed in contact with an eluent that flows across the stationary phase. The two separable components move different distances across the stationary phase by the eluent, and thus are effectively separated as desired. The separated components are then detected and analyzed using Raman spectroscopy. Detection can be facilitated by the adding SERS active nanoparticles, such as Au colloidal nanoparticles, to each location to which a separated component of the mixture has traveled along the stationary phase. 2D periodic pores with sub-micron diameters on diatomaceous earth enable guided-mode resonances (GMRs) of photonic crystals, which has similar effect with the diatom biosilica that has been used previously. For additional information, see, X. Kong et al., Optofluidic Sensing from Inkjet-Printed Droplets: the Enormous Enhancement of Evaporation Inducted Spontaneous Flow on Photonic Crystal Biosilica, Nanoscale, 8, 17285-17294 (2016), which is incorporated herein by reference in its entirety.

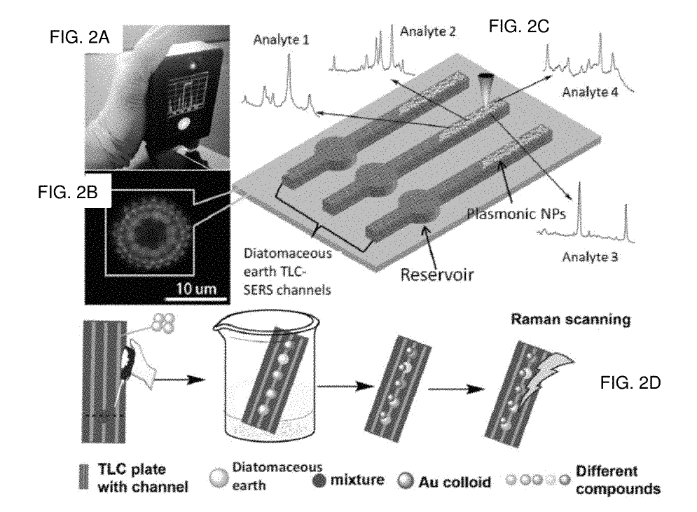

In another embodiment and with reference to FIGS. 2A-2D, one disclosed system comprised TLC-SERS channelized chips (FIG. 2C) and a commercial hand-held Raman spectrometer (FIG. 2A). On-chip chromatography is conducted first by adding a suitable amount of a test sample to the reservoir of the TLC-SERS chip. The test sample can be added as a solid, but most typically is dissolved in a solvent, and then added to reservoir. The sample includes at least one component that is detectable by Raman spectroscopy, such as SERS. For certain embodiments, microliter amounts of sample are added to the reservoir, such as from 0.1 μl to 50 μl, and more typically 0.1 μl to about 5 μl, although a person of ordinary skill in the art will appreciate that the amount of sample added will vary. The TLC-SERS chip is then edge-dipped into an eluent for a period of time, generally a few minutes, sufficient to allow the eluent to move by capillary action along the length of the channel. This process effectively separates different chemical compounds from the mixture applied to the reservoir (FIG. 2C) along the micro-porous diatomaceous earth channels as illustrated in FIG. 2D. In one embodiment, the reservoir was approximately 0.1 millimeter to 2.0 millimeters in diameter. After separation, a hand-held Raman spectrometer scans the micro-porous diatomaceous earth channels to obtain the entire SERS spectra of all chemical species in the testing sample that separated along the channel. And, again, Raman analysis can be facilitated by adding SERS active nanoparticles to each location to which a separated component of the mixture has traveled along the stationary phase. Unlike any presumptive and confirmative test, this facile lab-on-a-chip TLC-SERS technology provides universal, label-free identification of a broad range of chemical species with ultra-high sensitivity and specificity.

B. Detectable Target Molecules

Embodiments of the device, system and method described herein provide a universal analytical tool useful for many civilian and defense sensing applications. The device, system and method can be used to sense, label-free, multiple compounds in parallel as long as the target molecules have signature Raman spectra. Detectable target molecules include, but are not limited to:

Food adulterants and/or hazardous ingredients such as antibiotics, dyes, pesticides, hormones, contaminants, or combinations thereof, for example, Sudan dye, histamine, carbendazim, or a combination thereof;

Explosives, such as TNT, DNT, ammonium nitrate, TATP, PETN, RDX, TNB, DNAN, HMTD, etc.;

Chemical warfare agents, such as sarin (GA), tabun (GB), VX, mustard gas (HD), 2-chloroethyl ethyl sulfide, triphenyl phosphate, dimethyl methyphosphonate, etc.;

Illicit drugs and toxicants, such as cocaine, heroin, morphine, codeine, nicotine, mefenorex, pentylenetetrazole, pemoline, caffeine, erythropoietin (EPO), hydrocodone, amphetamines, benzodiazepines species, etc.;

Pollutants, such as carbendazim, imidacloprid, acetamiprid, phoxim, boscalid, buprofezin, myclobutanil, benzene, pyridine, xylene, formaldehyde, perchloroethylene, toluene, etc.;

Fire accelerants, such as polycyclic aromatic hydrocarbons (PAHs) in gasoline; and

Gunshot residues, such as ethyl and/or methyl centralite in smokeless powder.

C. Characterization and Evaluation of SERS-Active Nanoparticles

Certain disclosed embodiments concern using SERS-active nanoparticles to enhance target molecule detection. A person of ordinary skill in the art will appreciate that a number of different nanoparticles are suitable for use in the disclosed methodology, including by way of example, and without limitation, gold (Au) nanoparticles (NPs), silver (Ag) nanoparticles, copper (Cu) nanoparticles, aluminum (Al) nanoparticles, or combinations thereof. Certain disclosed embodiments preferably used gold colloidal nanoparticles. Certain suitable nanoparticles may be purchased, or they may be prepared according to methods known in the art. Example 1 below provides one exemplary embodiment of a method for making suitable Au nanoparticles.

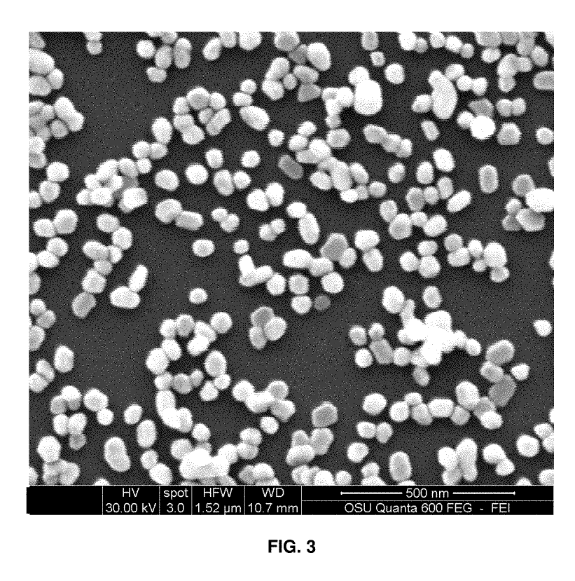

Scanning electron microscopy (SEM) and UV-vis spectroscopy can be used to characterize the morphology and properties of the nanoparticles (NPs). FIG. 3 is a scanning electron micrograph (SEM) of gold nanoparticles that were made for use with the present invention. FIG. 3 illustrates that the gold nanoparticles were substantially spherical and had a substantially uniform size distribution with particle diameters estimated to be about 60 nm.

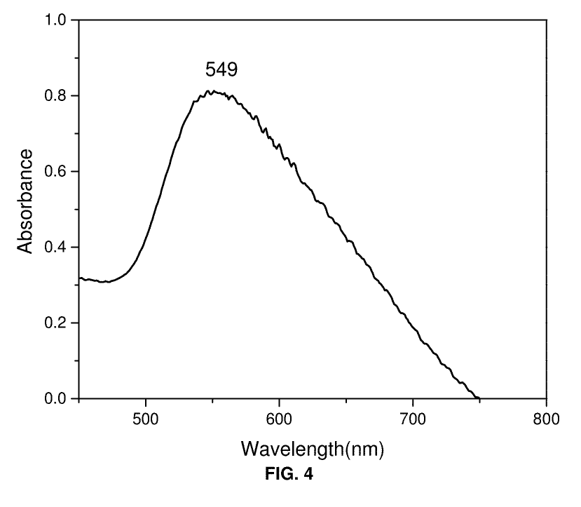

FIG. 4 is a UV-vis spectrum of a localized surface plasmon resonance (LSPR) band at 545 nm with a narrow bandwidth for Au colloidal nanoparticles prepared for use with the present invention. These values correspond to relatively uniform, mono-dispersed Au colloidal nanoparticles having diameters of approximately 50-60 nm, which is a preferred size range for some disclosed embodiments. In other embodiments, the colloids can range from 10 nm to as large as 200 nm. The concentration of Au nanoparticles was estimated to be approximately 1×10⁻¹⁰ M, by using Lambert's law based on UV-vis spectroscopy with a molar extinction coefficient of 3.4×10M⁻¹ cm⁻¹.

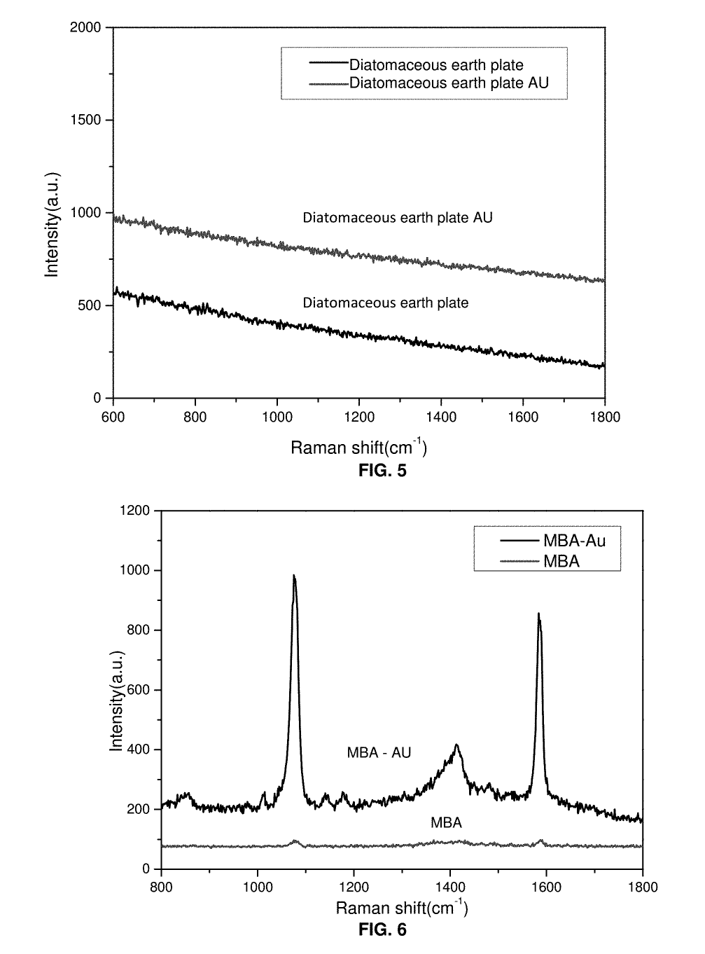

FIGS. 5-7 establish that SERS analysis is substantially enhanced by using gold colloidal nanoparticles. This was verified by obtaining Raman spectra of diatomaceous earth TLC plates both with and without added Au nanoparticles. FIG. 5 provides SERS spectra of diatomaceous earth TLC plates with and without the application of Au nanoparticles. FIG. 5 establishes that the intensity of the Raman signal was increased from about 550 a.u. to at least about 950 a.u. by application of Au nanoparticles. This effect was further verified by obtaining SERS spectra of an MBA solution, a typical Raman probe molecule, with and without the addition of Au nanoparticles to MBA TLC spots. Applying SERS active nanoparticles to detectable targets substantially increases the intensity of the Raman signal. FIG. 6 establishes that the Raman intensity increased from less than 100 a.u. to at least about 1,000 a.u. by applying the Au nanoparticles to the MBA spots prior to Raman analysis.

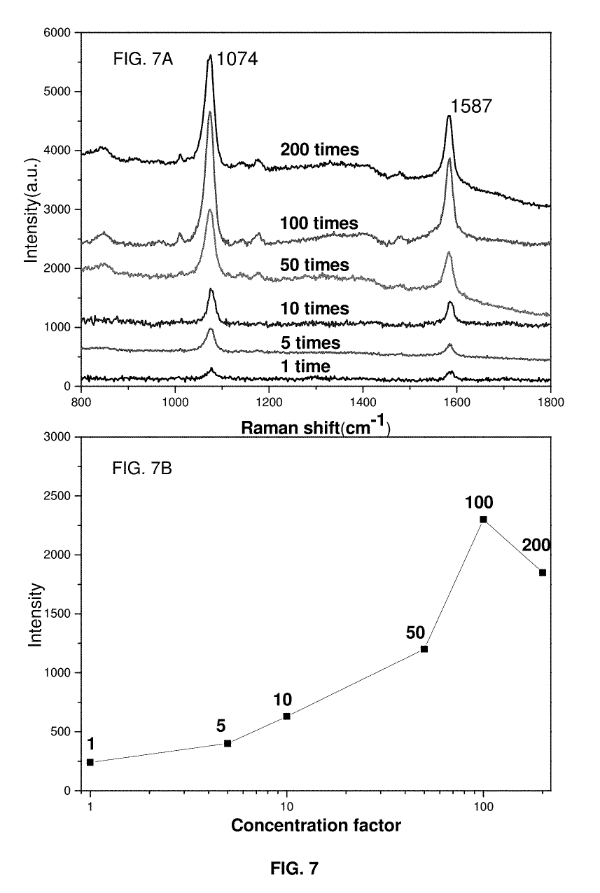

The concentration of metallic colloids also may affect Raman signal enhancement. FIG. 7A provide SERS spectra of 100 ppm mercaptobenzoic acid (MBA) on diatomaceous earth TLC plates by applying 2 μl casted gold colloids at six different concentrations. SERS spectrum intensity of MBA clearly increased as the colloidal gold concentration increased 100 times relative to the original concentration. However, the measured SERS intensity decreases if the colloidal gold concentration increased more than 100 times relative to the original concentration (FIG. 7B). Without being bound by a theory of operation, it appears that a higher concentration of metallic nanoparticle colloids results in accretion of denser monolayer coverage, which increases the SERS signals. However, if the concentration exceeds that required for monolayer formation, multilayer nanoparticle accumulation reduces the intensity of the SERS signals. In some embodiments, nanoparticles are used at from 50-fold to 150-fold concentrations, such as from 90-fold to 120-fold concentration. And in certain embodiments, a 100-fold concentrate (1×10⁻⁸ M) of gold colloids were selected for subsequent analyses.

D. Microstructures of the Diatomaceous TLC Plate

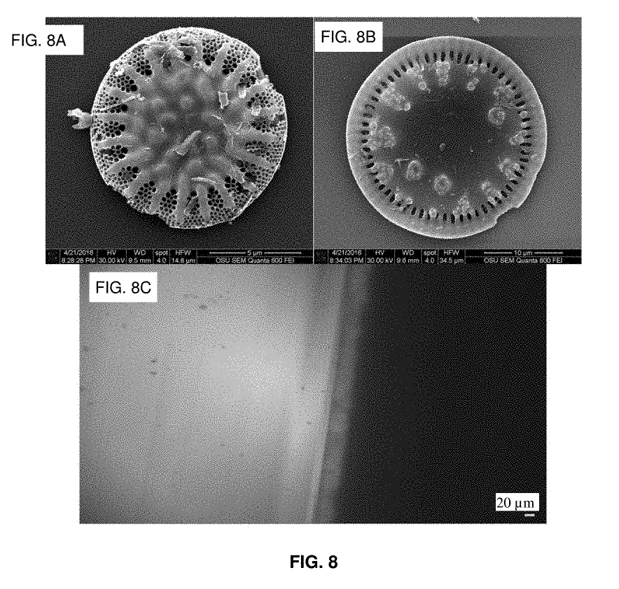

For some disclosed embodiments, SEM was used to characterize the morphology of the diatomaceous earth. FIG. 8 provides SEM images establishing that the main component of commercial diatomaceous earth is disk-shaped with periodic pore structures (FIGS. 8A and 8B). The size distribution of diatomaceous earth typically ranges from greater than 0 μm to at least 50 μm, more typically from 10 μm to about 30 μm. Diatomaceous earth is applied to a TLC plate by an acceptable method, such as spin coating, and operates as the stationary phase on the TLC plate. The thickness of the diatomaceous earth as applied to a substrate, such as a glass plate, can vary. Typically the applied diatomaceous earth has a thickness that varies from greater than 0 to at least about 100 nanometers, and more typically is from about 5 μm to about 20 μm for disclosed embodiments. Thickness of deposited diatomaceous earth on glass was monitored by optical microscopy as shown in FIG. 8C. In some embodiments, the thickness of the diatomaceous earth was substantially uniform and measured to be about 20 μm, which is much thinner than the commercial silica gel TLC plate of 60-100 μm. In some embodiments, a thinner layer is may be preferable as decreasing stationary phase thickness may increase Raman signal sensitivity, whereas Raman signal intensity may decrease as the stationary phase thickness increases.

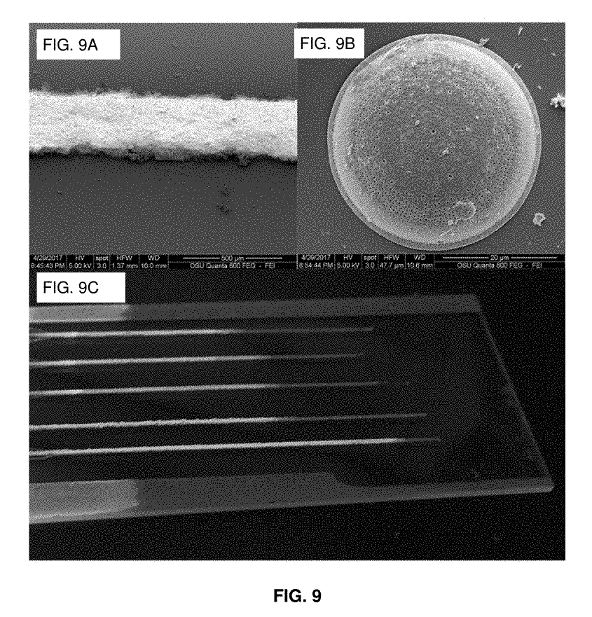

Certain disclosed embodiments concern forming microchannels made from or comprising diatomaceous earth, as discussed in more detail below. The morphology of a disclosed diatomaceous earth porous microchannel was characterized by SEM and is shown in FIG. 9A. Diatomaceous earth porous microchannels typically range in width from approximately 100 μm to 1.0 mm. In one embodiment, the width of the porous microchannel was approximately 400 μm. For certain embodiments, the porous microchannel was fluidly associated with a reservoir. The reservoir dimensions can also vary as will be understood by a person of ordinary skill in the art, but typically range from 0.1 mm to several millimeters in diameter. For certain working embodiments the reservoir had a diameter of about 1 millimeter. The porous microchannels (diatomaceous earth channels) mainly comprised disk-shaped, diatomaceous earth biosilica. The morphology of diatomaceous earth biosilica used for certain disclosed embodiments is provided by FIG. 9B. Diatomaceous earth biosilica has 2D periodic pores with sub-micron diameters that enable guided-mode resonances (GMRs) of photonic crystals, which has similar effect with the diatom biosilica that has been used previously.

In order to verify fluid migration in the 3D porous microchannel, 100 ppm pyrene solution was used as fluid sample. After migration, the porous microchannel was illuminated under UV light, and the resulting image is shown in FIG. 9C. The brighter color was observed in contrast with the glass, which indicated that the 3 D porous microchannel structure could be successfully used for fluid migration.

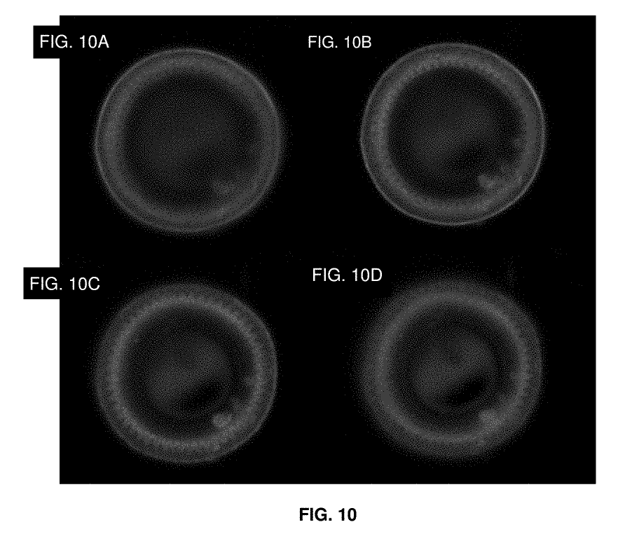

In order to verify the photonic crystal effect of diatomaceous earth, the optical microscopic image of a single diatom is shown in FIGS. 10A-10D. The light pattern is produced by a high order diffraction of the photonic crystals. Therefore, the diatom nanostructures provide certain photonic crystal properties. The highly porous structure and uniform diatom pore size has lower fluid flow resistance, which enables more homogenous fluid flows into the pores of diatom. The eluent therefore migrates more smoothly and uniformly on the surface of a stationary phase comprised of diatomaceous earth, such as may be provided by a plate or a porous microchannel, during the development process.

When collecting SERS spectra of an analyte(s) on TLC plates, the background SERS signals originating from the blank stationary phase should be determined first. Therefore, it is necessary to investigate the SERS signals from different TLC plate matrices. The measured results indicate that both the diatomaceous earth and the silica gel TLC plates exhibit a weak, broad spectral background, with no obvious Raman peaks observed. Thus it is conceivable to use diatomaceous earth TLC plates for SERS detection.

E. SERS of Single Components and Mixtures

Polycyclic aromatic hydrocarbons (PAHs) are a class of organic compounds comprising two or more aromatic or heterocyclic rings. PAHs pose serious health and environment risks. Unfortunately, the low binding affinity between PAHs and metallic substrate surfaces blocks efficient SERS detection of PAHs from mixtures as the spectra from co-existing components interfere with the spectrum from the PAHs.

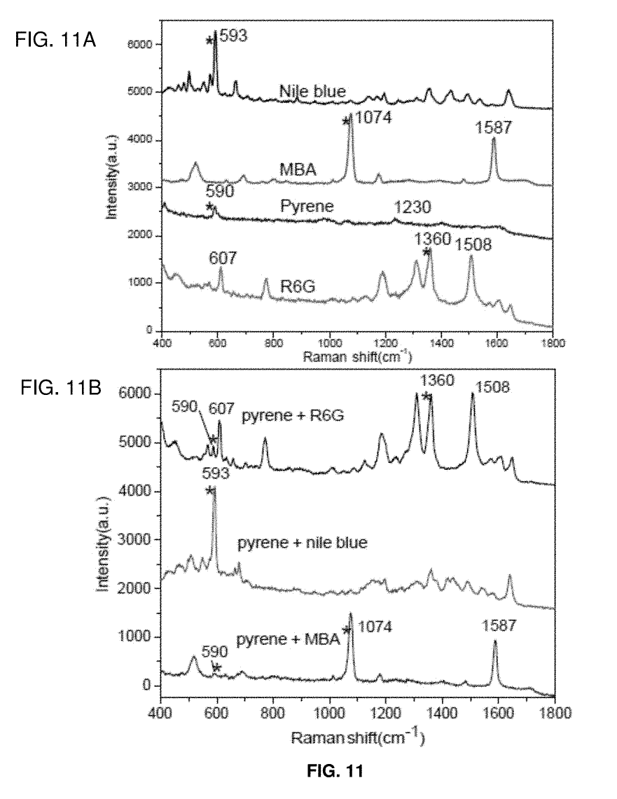

Certain disclosed embodiments address this issue. To overcome this problem, pyrene was mixed with three Raman probe molecules [mercaptobenzoic acid (MBA), R6G and Nile blue (NB)] respectively to form different mixtures. FIG. 11A provides SERS spectra of these pure substances. Specifically, for pyrene, the peak at 590 cm⁻¹ is assigned to the skeletal stretching vibration and 1230 cm⁻¹ and is associated with the C—C stretching/C—H in-plane bending of pyrene. For MBA, the peaks located at 1074 and 1587 cm⁻¹ are associated with C—C ring-breathing modes. For R6G, the peak at 607 cm⁻¹ is associated with C—C—C ring in plane vibrations, while the peaks at 1360 and 1508 cm⁻¹ are associated with aromatic C—C stretching vibrations. For NB, the peak at 594 cm⁻¹ is assigned to in-plane deformation vibration of the NB. Asterisks are assigned to Raman spectral peaks that uniquely identify the individual substances.

The SERS spectra of three mixtures (pyrene plus R6G; pyrene plus nile blue; and pyrene plus mercaptobenzoic acid) are provided by FIG. 11B. For Mixture 1 (Pyrene and MBA 1/1), the metallic surface coverage was dominated by MBA because of facile covalent bond formation between the Au nanoparticles and the mercapto group of MBA. Thus only a very weak Raman peak from pyrene was observed from the SERS spectra of mixture 1. For Mixture 2 (Pyrene and R6G 1/1), R6G is a typical Raman probe molecule because of its affinity with metallic surfaces and intense Raman signals. The in plane vibrations of R6G are located at 607 cm⁻¹, which are near the feature Raman peak of pyrene at 590 cm⁻¹, making it hard to distinguish the Raman peak of pyrene from the mixture due to the intense SERS signals from R6G. For Mixture 3 (Pyrene and NB 1/1), NB is another Raman probe molecule that is often used to evaluate the SERS performance of the substrates. The intense band located at 593 cm⁻¹ is usually used as the feature peak of NB in detection. But this peak overlaps with the main Raman peak of pyrene. Therefore, only molecule information of NB can be observed from the SERS spectra of mixture 3.

The chromatography performances of the diatomaceous earth TLC plates and commercial silica gel TLC plates were evaluated using the aforementioned three mixtures. A person of ordinary skill in the art understands that a suitable eluent must be selected to perform thin layer chromatography, and further that the present disclosure is not limited to a particular eluent solvent system. By way of example, and without limitation, suitable eluents include: aliphatic compounds, particularly alkyl solvents, such as C₅-C₁₀ alkyl solvents; aromatic compounds, such as toluene; alcohols, such as C₁-C₁₀ alcohols; heteroaliphatic compounds, such as ethers, esters and amines, particularly C₂-C₁₀ heteroaliphatic compounds; and combinations of such eluents. For the present TLC separation, a hexane and ethyl acetate (v/v=3:1) mixture was used as an eluent suitable for separating pyrene from the mixtures.

After separation, a suitable visualization method must be determined to visualize separated components. A person of ordinary skill in the art will appreciate that there are a number of different chemical and physical methods for visualizing mixture components separated by layer chromatography, and any such methods may be used with disclosed embodiments of the present invention. Solely by way of example and without limitation, acceptable visualization systems include: aniline phthalate, p-anisaldehyde—sulfuric acid, p-anisidine hydrochloride, anisidine phthalate, antimony (iii) chloride, antimony (iii) chloride, bromine/carbon tetrachloride, bromocresol green, bromthymol blue, chloranil reagent, chlorine/o-tolidine, copper sulfate/phosphoric acid, chromosulfuric acid, potassium dichromate/sulfuric acid, dichlorodicyanobenzoquinone, dichlorofluorescein, dichlorofluorescein/fluorescein sodium salt, 2,6-dichloroquinone-4-chloroimide, p-dimethylaminobenzaldehyde, p-dimethylaminobenzaldehyde/hydrochloric acid reagent (ehrlich's reagent), 2,4-dinitrophenylhydrazine, diphenylamine, s-diphenylcarbazone, 2,2′-diphenylpicrylhydrazyl, dithizone, dragendorff reagent, ethanolamine diphenylborate, erhlich's reagent (p-dimethylaminobenzaldehyde), emerson reagent (4-aminoantipyrine/potassium hexa-cyanoferrate (iii)), fast blue b reagent, ferric chloride/sulfuric acid, fluorescamine, formaldehyde/sulfuric acid, formaldehyde/phosphoric acid, furfural/sulfuric acid, gentian violet—bromine, gibb's reagent, iodoplatinate, iron (iii) chloride/potassium hexacyanoferrate/sodium arsenate (according to patterson & clements), lead tetraacetate/2,7-dichlorofluorescein, manganese/salicylaldehyde, mandelin's reagent (vanadium(v)/sulfuric acid), mercury (ii) chloride/diphenylcarbazone, mercury (ii) chloride/dithizone, 4-methoxybenzaldehyde/sulfuric acid/ethanol, methyl yellow, molybdatophosphoric acid, ninhydrin, ninhydrin/cadmium acetate, ninhydrin/pyridine/glacial acetic acid, nitric acid/ethanol, orcinol (bials reagent), paraffin oil, m-phenylenediamine, o-phenylenediamine—trichloroacetic acid, p-phenylenediamine—phthalic acid, phenylhydrazine sulfonate, phosphoric acid, phosphoric acid—bromine, bromate and 2 ml 25% hydrochloric acid, phosphomolydbic acid, phosphotungstic acid, pinacryptol yellow, rhodamine b, rhodamine 6 g, silver nitrate/hydrogen peroxide, sodium azide, sodium 1,2-napthaquinone-4-sulfonate (nzs reagent), sodium nitroprusside/hydrogen peroxide, sodium nitroprussate/potassium hexacyanoferrate (iii), stannic chloride, tetracyanoethylene—tcne reagent, tetranitrodiphenyl, tetrazolium blue, thymol/sulfuric acid, tin (iv) chloride, o-tolidine, diazotized, p-toluenesulfonic acid, trichloroacetic acid, trifluoroacetic acid, tungstophosphoric acid, urea/hydrochloric acid, vanadium (v)/sulfuric acid, ammonium monovanadate (ammonium metavanadate)/sulfuric acid reagent, vanadium pentoxide/sulfuric acid reagent, vanillin/potassium hydroxide, vanillin/sulfuric acid, potassium dichromate/sulfuric acid (chromosulfuric acid), UV light, iodine, or a combinations thereof.

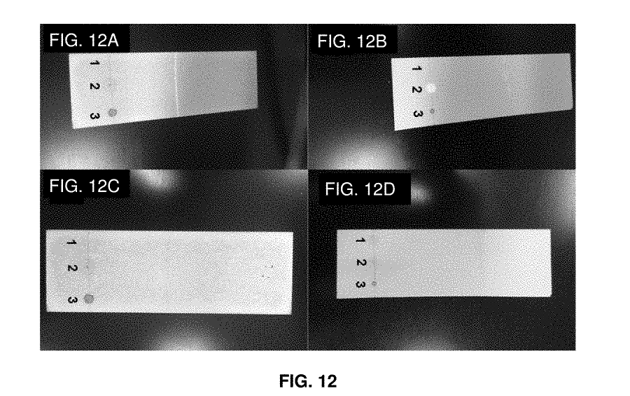

For the TLC results presented by FIGS. 12A-12D, a UV lamp and iodine colorimetry were used to detect different analyte spots corresponding to various substances as shown by the digital images of FIGS. 12A-12D. Pyrene traveled at faster speeds and located further from the original dropping points because of the low molecular polarity. Three different types of mixtures have been successfully separated as shown in FIGS. 12A-12D. The retention factor (Rf) (equal to the ratio of distance migrated by the component and solvent on TLC plate) values of the mixing points were obtained by the UV light scanner and iodine colorimetric, which are 0/0.8 on silica gel plate and 0/0.9 on diatomaceous earth plate. Pyrene moved farther from the origin spot due to its lower polarity compared with other components in the mixture. Diatomaceous earth TLC plates according to the present invention provide a substantially improved separation capability compared to commercial TLC plates under the same separation conditions.

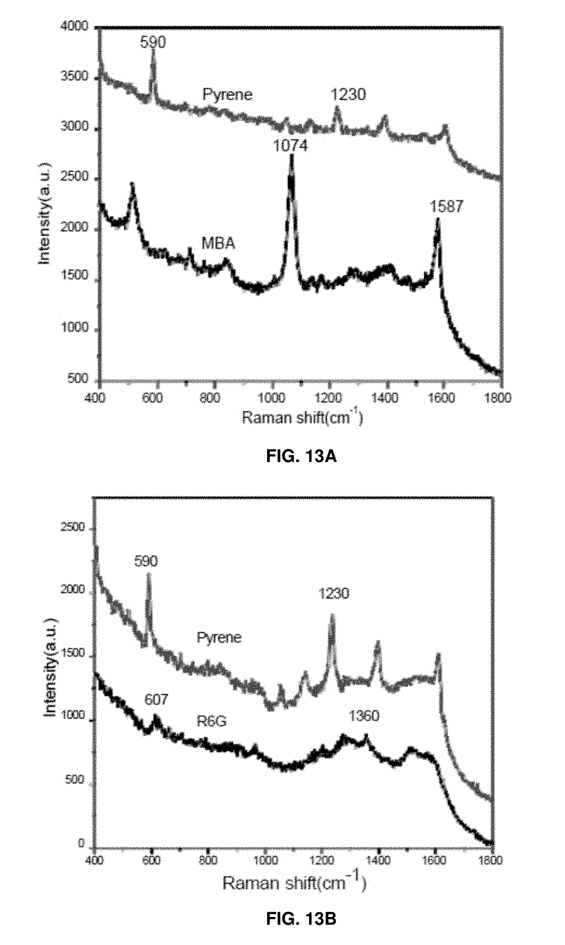

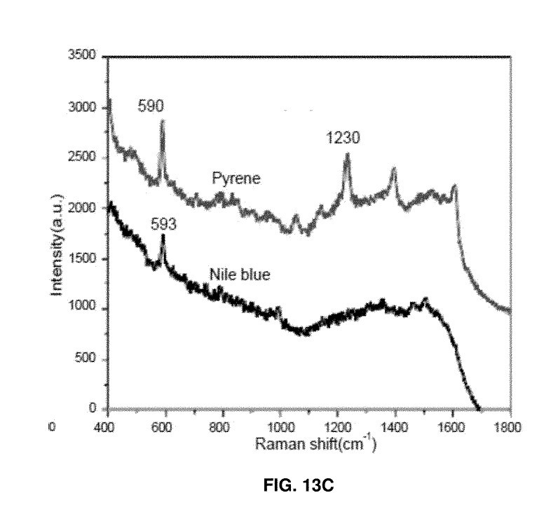

SERS spectra of different spots separated on a diatomaceous earth plate by TLC are shown in FIGS. 13A-13C. The feature peaks of pyrene at 590 cm⁻¹ and 1230 cm⁻¹ are easily observed. This establishes that the diatomaceous earth plate can be used successfully as a stationary phase in a TLC-SERS method.

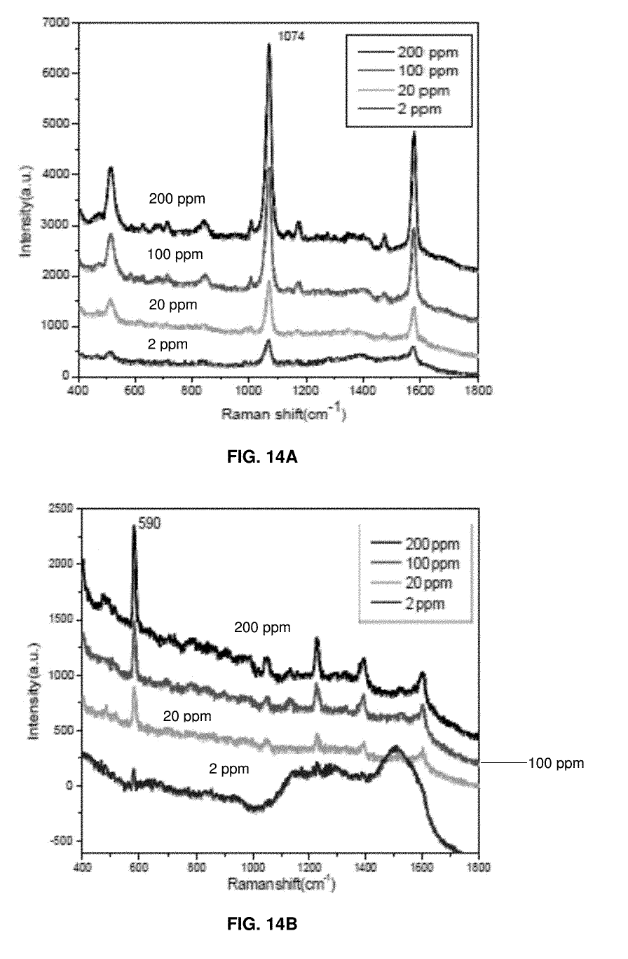

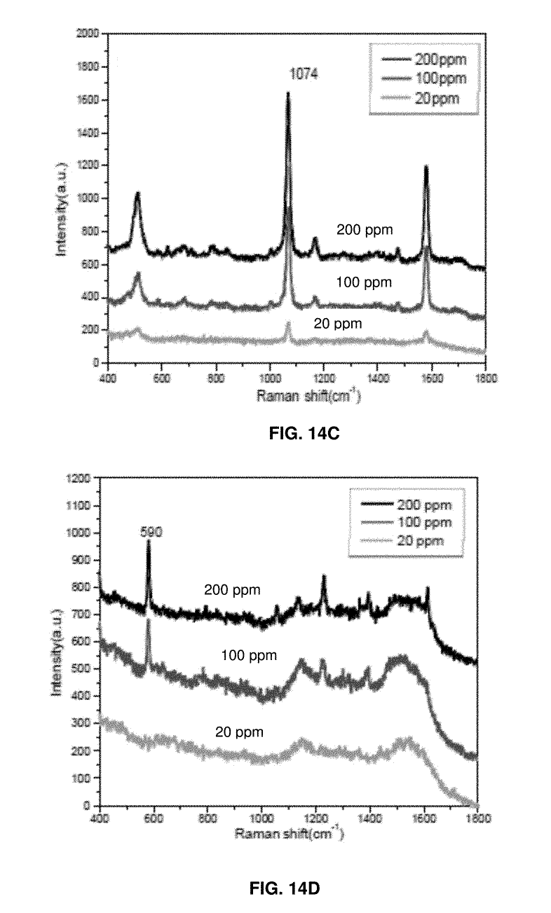

SERS spectra obtained from a diatomaceous earth TLC plate (FIGS. 14A and 14B) were compared to a commercial silica-gel TLC plate (FIGS. 14C and 14D). In FIGS. 14C and 14D all the characteristic bands of MBA and pyrene exhibited an incremental decrease in intensity, corresponding to a decrease in mixture concentration, relative to that provided by the diatomaceous earth TLC plate (FIGS. 14A and 14B). The lowest detection limit from pyrene/MBA mixture is between 20 and 100 ppm on the commercial TLC plate, and below 2 ppm on the diatomaceous earth TLC plate.

These results demonstrate a substantial improvement in intensity when using diatomaceous earth TLC plates relative to commercially available TLS plates, such as an intensity increase of at least 1 times up to at about 70 times, and more typically an intensity increase of about 50 times. Without being bound to a particular theory of operation, this intensity increase may be attributed to two contributions from the diatomaceous earth plate. First, in most TLC-SERS methods, metallic nanoparticles are casted onto pre-separated TLC component spots. The SERS spectra collected from each spot are produced solely by target molecules at the surface of the TLC plate. This means that overall sensitivity will be compromised because a substantial portion of analyte molecules inside the TLC plate stationary phase material cannot be detected. The thickness of the diatomaceous earth TLC plates fabricated by spin coating according to the present invention is substantially thinner than that provided by commercially available TLC plates. For example, commercial silica plates having stationary phase material layer thicknesses of from about 60 μm up to about 100 μm. Diatomaceous earth silica plates made according to the present disclosure typically have a substantially thinner layer, such as from greater than 0 μm to at least 100 μm, preferably greater than zero to about 50 μm, greater than 5 μm to about 50 from 10 μm to 30 μm, and typically from about 15 μm to about 20 μm, with certain working embodiments having a stationary phase material layer thickness of about 20 μm, i.e. about one third of the thickness of the commercial silica-gel TLC plate. The thinner diatomaceous earth layer will achieve much higher sensitivity. Second, diatomaceous earth consists of fossilized remains of diatoms, a type of hard-shelled algae. The two dimensional (2-D) periodic pores on diatomaceous earth with hierarchical nanoscale photonic crystal features. The hybrid photonic-plasmonic modes were formed when Au NPs deposited onto the surface of diatomaceous earth, which will further increase the local electric field of Au NPs, and the additional enhancement of SERS obtained. Previous studies have proven that on diatoms biosilica through theoretical calculation and experimental result.

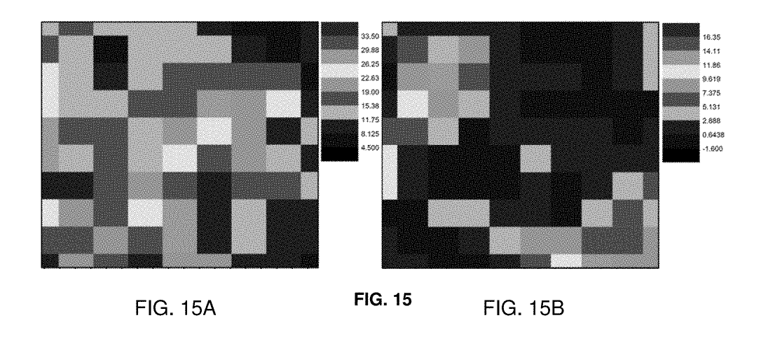

Mapping images of the Raman signals visualized the distribution of analytes on the TLC plate. FIG. 15 provides Raman mapping images of MBA (10 ppm) on diatomaceous earth (FIG. 15A) and silica gel (FIG. 15B) TLC plates. MBA provides an intense Raman peak at 1074 cm⁻¹, which is assigned to the ring-breathing modes. The SERS mapping image was recorded using the integrated peak intensity at 1060-1090 cm⁻¹. Diatomaceous earth TLC plates according to the present invention showed a much stronger and more uniform SERS signals of MBA than the silica gel TLC plate did. The highly porous structure and larger pore size of the diatomaceous earth has lower flow resistance, which enables more liquid to flow into the pore, and the eluent migrates in contact with substantially the entire surface of the stationary phase during the TLC development. For the commercial silica gel TLC plate, eluent flows mainly through gaps between silica gel particles, and the analytes located on the interparticle area.

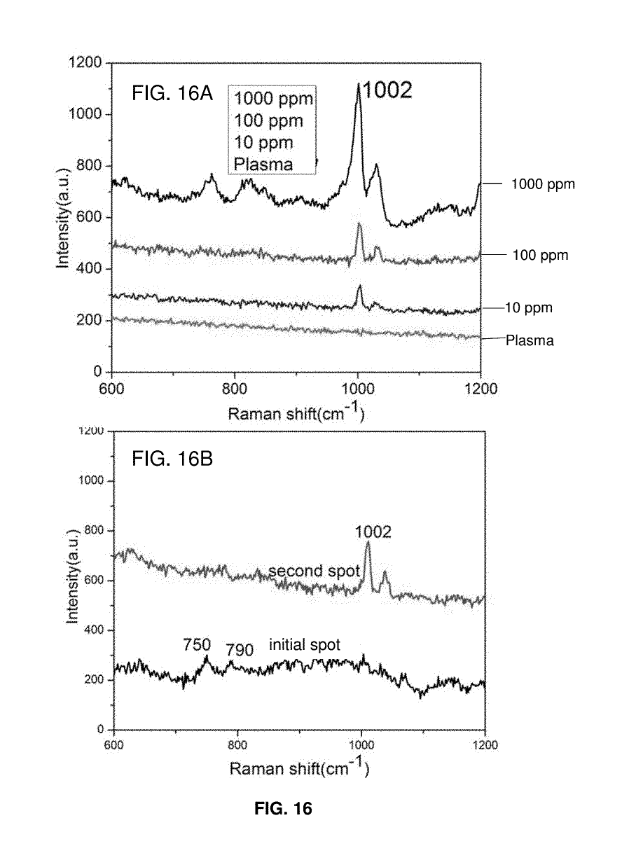

SERS detection of analytes in biofluid is complex. Moreover, the high saline concentration can make metal NPs in solution (i.e., metallic colloids) unstable. Certain disclosed embodiments of the present invention address this problem by using TLC-SERS for on-site detection of biogenic amines from plasma. This approach has been proved using an exemplary process comprising using ammonium hydroxide and ethanol (v/v=1:1) as an eluent for separating phenethylamine (PEA) from plasma. Some biomolecules, such as albumin in plasma, cannot diffuse on the TLC plate due to the high molecular weight. FIG. 16A provides SERS spectra obtained using this process. The Raman peak at 1002 cm⁻¹ was assigned to the phenyl ring breathing vibration of PEA. The lowest detection limit for PEA/plasma was 10 ppm on the diatomaceous earth TLC plate. As a comparison, there were no detectable SERS signals of PEA on a silica gel TLC plate even when the concentration of PEA was 100 ppm. This result demonstrates that disclosed embodiments provide a substantial improvement in the level of analyte detection, such as at least 2 times, or at least 5, such as at least 10, and in certain embodiments, more than a 50 times improvement in the level of detection of an analyte using the diatomaceous-earth-based TLC plate compared to commercially available silica-gel TLC plates.

It is difficult to obtain a SERS signal of proteins that have no conjugated chromophore. Fourier transform infrared spectroscopy (FTIR) was employed to verify the protein in plasma during a TLC process. IR peaks were observed at 1645 cm⁻¹ and 1540 cm⁻¹, and these peaks are assigned to amide I and amide II bands of protein in plasma, and an IR peak at 1585 cm⁻¹ was assigned to C═C stretching vibration of PEA. To more effectively show the performance of TLC-SERS, miR21cDNA was added into the plasma (5×10⁻⁶ M). SERS spectra of DNA was observed at the initial spot after the TLC separation as shown in FIG. 16B. The peaks at 750 cm⁻¹ and 790 cm⁻¹ were assigned to the ring breathing modes of thymine and cytosine, respectively. This is the first time that a TLC-SERS method was employed to detect analytes from plasma.

Accordingly, the above results demonstrate that diatomaceous earth is a highly effective material for on-chip TLC and SERS plates for both separating and detecting components from mixtures comprising SERS detectable components, as exemplified by separating pyrene from mixtures comprising Raman detectable probes, and PEA from plasma. This method provides a simple, rapid and cost-effective route for separating and detecting analytes from mixtures. The experimental results demonstrate more than 10 times improvement of sensitivity by using the diatomaceous earth-based TLC plate compared to the commercial silica-gel TLC plate. This facile porous diatomaceous earth base TLC-SERS method is convenient, fast, cost effective, sensitive, and has potential application for on-site monitoring of pollutants and toxins in environments and identifying illicit drugs in biofluid.

In yet other embodiments, described further below, the sensitivity of the TLC-SERS plates is further enhanced by using microchannels formed from, or comprising, diatomaceous earth. These embodiments improve pre-concentration and separation of molecules in a complex mixture.

In general, the intensity of SERS I_{SERS(vs)} can be estimated with the following equation:

I_{SERS}(V_{S})∝N_{M}×|A(V_{L})|²×|A(V_{S})|²×δ_{ads}^{R}

where N_{M} is the number of molecules involved in the SERS measurement, δ_{ads}^{R} is the Raman cross section of the molecule that is being detected, and A(V_{L}) and A(V_{S}) are the electrical field enhancement factors at the extinction laser and Stokes frequency for the Raman signal enhancement. These parameters usually are intrinsic factors which are nearly constant for the same SERS substrate and the target molecule other than NM. Plasmonic nanoparticles may be dispensed onto the analyte spots after chromatographic separation of a mixture into the components. The SERS spectra collected from each spot solely obtains from the target molecules at the surface of the chromatography chip. Accordingly, the overall SERS intensity depends on the amount of target molecule present at the surface of a particular chromatography chip. Thinner diatomaceous earth layers as disclosed for use with the present invention achieve higher analyte concentration at the surface of the chromatography plate. The porous microchannel described herein further confines the liquid flow within a narrow range compared with a normal chromatography chip. This enables pre-concentration target molecule on the surface of a porous microchannel.

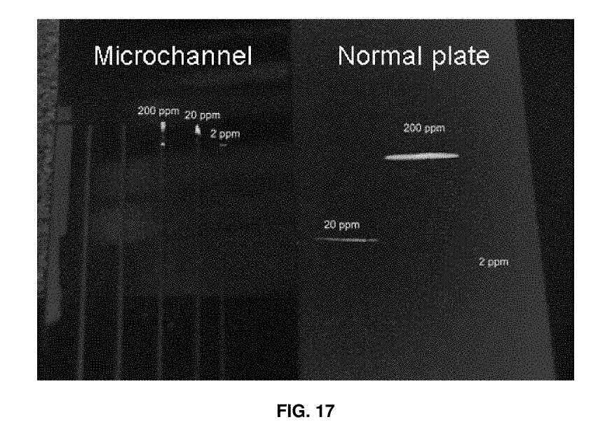

The analyte pre-concentration effect of an exemplary diatomaceous earth porous microchannel was demonstrated by fluorescence microscopy and spectra. First, 0.2 μL of a solution comprising 200 ppm, 20 ppm and 2 ppm pyrene was dispensed onto an exemplary diatomaceous earth porous microchannel chip and a normal diatomaceous earth chromatography chip. After eluent migration, the substrate was illuminated by a UV laser, as shown in FIG. 17, allowing observation of the fluorescence spots on the two different chips. For samples comprising 20 ppm pyrene, the fluorescence spot from an exemplary porous microchannel chip was substantially brighter than that from a normal chromatography chip. When the concentration of pyrene was reduced to 2 ppm, the fluorescence spot on the porous microchannel was still readily observable, and yet no fluorescence spot was observed on the normal chromatography chip.

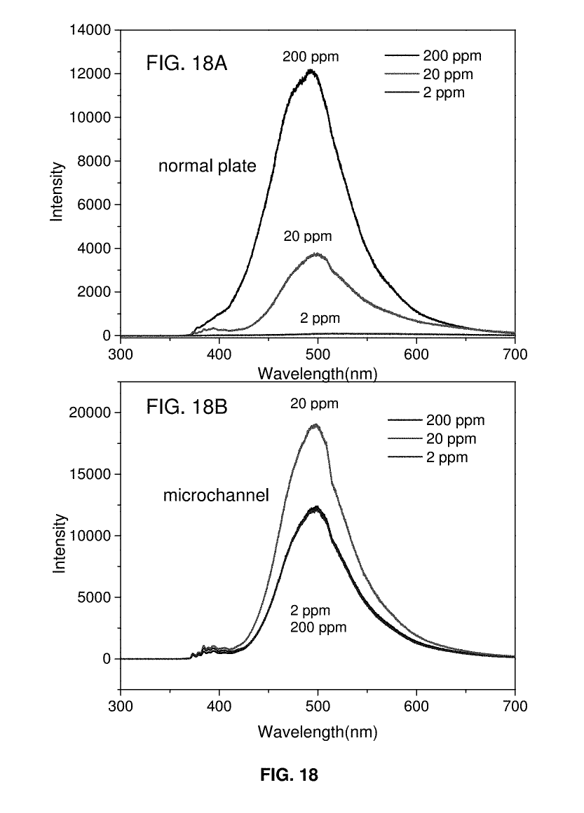

This target molecule pre-concentration effect was also confirmed by fluorescence spectra as shown by FIGS. 18A and 18B. The sample used to acquire the fluorescence spectra was the same as those for fluorescence imaging. As demonstrated by FIG. 18A, the intensity of pyrene fluorescence spectra decreased as the pyrene concentration decreased. Only weak pyrene fluorescence spectra were obtained when the pyrene concentration was reduced to 2 ppm. In contrast, the pyrene fluorescence spectra produced following separation using a porous microchannel according to the present invention is provided by FIG. 18B. The 2 ppm pyrene spot still produced an intense fluorescence signal. The small amount of the pyrene processed using the porous microchannel had a substantially higher fluorescence intensity than that from 20 ppm pyrene on a normal chromatography chip. More specifically, the pyrene fluorescence had an intensity of 12,000+ using a porous microchannel and method according to the present invention, whereas the normal plate produced a sample having virtually no fluorescence. This demonstrated that the narrow microchannel had a pre-concentration effect on this target molecule.

F. Fabrication of Exemplary Diatomaceous Earth Porous Microchannels

Diatomaceous earth substrates were fabricated by applying diatomaceous earth to a substrate, such as a glass plate or slide. The diatomaceous earth may be, and typically is, dried at a suitable temperature and for a suitable period of time to obtain substantially dry material, such as dried at 150° C. for 6 hours in an oven. The dried diatomaceous earth can be applied to the glass plate using any of a number of effective processes, including spray coating, spin coating, doctor blade application, etc., with spin coating being used to apply diatomaceous earth to glass slides for this particular embodiment. After cooling to room temperature, an appropriate amount of diatomaceous earth was dispersed in a suitable fluid for spin coating. Suitable dispersions typically comprise from 0.1 to 1.0 gram diatomaceous earth/milliliter of dispersing fluid, more typically from 0.5 to 0.6 grams diatomaceous earth/milliliter of dispersing fluid. For an exemplary working embodiment, 11.55 g of diatomaceous earth was first dispersed in 20 mL (0.575 gram diatomaceous earth/milliliter dispersion fluid) of a 0.4% aqueous solution of carboxymethyl cellulose to form a deposition dispersion. The deposition dispersion was then deposited onto the glass plate or slide, such as by spin coating at 1300 rpm for 20 seconds, and the plate or slide is allowed to dry.

Microchannels comprising the applied diatomaceous earth are then formed on the plate or slide using any suitable method, including masking, patterning, selective material removal, etc. Hierarchically porous photonic crystal biosilica microchannels were fabricated using a tape-stripping method. Glass slides were spin coated with diatomaceous earth and covered by an adhesive tape. 400 μm wide channels were then made using a razor blade to cut through the tape after spin-coating with diatomaceous earth. The tape was then gently removed, leaving a 400×30 μm² diatomaceous earth channel array formed on the glass substrate. These μDADs were the dried and activated at 110° C. for 3 hours to improve diatomaceous earth adhesion to the glass slides.

G. Microfluidic-SERS Method Using Porous Microchannels

The on-chip chromatography-SERS sensing method was designed for ultra-sensitive detection of analytes from mixtures or complex biofluid samples. First, a liquid sample comprising a target or analyte is applied to a sample reservoir formed on one end of a microchannel. For example, for certain embodiments, a 0.2 μL liquid sample was spotted onto the reservoir (circled region) of the μDAD. After drying in air, a bottom edge of the μDAD was immersed in a suitable eluent. The eluent migrates along the porous channels towards the opposite end of the μDAD. After migration, the μDAD is removed from the solvent and dried in air, although samples also could be dried under an inert atmosphere, such as nitrogen, if beneficial. Analytes in the mixture separate as fluid migration proceeds along the microchannel and form analyte spots at different locations along the microchannel.

The separated spots then need to be detected, and preferably visualized, by a suitable process. For particular exemplary embodiments according to the present invention, separated analyte spots along the porous channels typically were illuminated by UV light, such as ultraviolet illumination at 380 nm wavelength, and visualized by iodine colorimetry.

Nanoparticles may then be applied to the spots directly to facilitate SERS analysis. For example, 2 μL of gold nanoparticles (Au NPs) in solution were directly applied to the separated analyte spots. A Horiba Jobin Yvon Lab Ram HR800 Raman microscope equipped with a CCD detector was used to acquire the Raman spectra, and a 50× objective lens was used to focus the laser onto the SERS substrates. A 785 nm laser was chosen as the excitation wavelength, and the laser spot size was 2 μm in diameter. The confocal pinhole was set to a diameter of 200 μm. SERS mapping images were recorded with a 20×20-point mapping array. Images were collected using the DuoScan module with a 2.0 μm step size, 0.5 second accumulation time, and within the Raman spectral range from of 500 cm⁻¹ to 1600 cm⁻¹. The acquired data was processed with Horiba LabSpec 5 software. Fluorescence spectra were then acquired. See, Guerrero, A. R.; Aroca, R. F., Surface-Enhanced Fluorescence with Shell-Isolated Nanoparticles (SHINEF), Angewandte Chemie International Edition 50, 665-668 (2011), which is incorporated herein by reference, for additional information concerning acquiring Raman spectra. Briefly, the Raman system was focused on the diatom surface. For example, a 50× objective lens of a Horiba Jobin Yvon Lab Ram HR800 Raman system with a 325 nm UV laser line was focused on an appropriate portion of the diatom surface.

### II. Examples

The following examples are provided to illustrate certain features of exemplary working embodiments. A person of ordinary skill in the art will appreciate that the scope of the present invention is not limited to the features of these working embodiments.

A. Materials and Methods

Tetrachloroauric acid (HAuCl₄) was purchased from Alfa Aesar. Trisodium citrate (Na₃C₆HsO₇), anhydrous ethanol, ammonium hydroxide (NH₃.H₂O), hexane and acetate were purchased from Macron. Celite209 (diatomaceous earth), cellulose, pyrene, 4-mercaptobenzoic acid (MBA), Nile blue, plasma, cocaine and phenethylamine (PEA) were obtained from Sigma-Aldrich. Rhodamine6G (R6G) was purchased from TCI. The chemical reagents were all analytical grade. Water used in all experiments was deionized and further purified by a Millipore Synergy UV Unit to a resistivity of ˜18.2 MΩ cm.

B. Instruments

UV-vis absorption spectra were recorded on NanoDrop 2000 UV-Vis spectrophotometer (Thermo Scientific) using quartz cells of 1 centimeter (cm) optical path. FTIR attenuated total reflectance (ATR) infrared spectra were recorded on a Nicolet 6700 Fourier transform infrared (FT-IR) spectrometer (Thermo Scientific) and Smart iTR diamond ATR accessory fitted with a liquid nitrogen-cooled MCT detector. Scanning electron microscopy (SEM) images were acquired on an FEI Quanta 600 FEG SEM with 15-30 kV accelerating voltage. The microscopy images were acquired on the Horiba Jobin Yvon Lab Ram HR800 Raman microscope using a 50× objective lens.

### Example 1

This example details one embodiment for preparing and characterizing gold nanoparticles (Au NPs). The glassware used for nanoparticle (NP) synthesis was cleaned with aqua regia (HNO3/HCl, 1:3, v/v), followed by rinsing thoroughly with Milli-Q water. Au NPs with an average diameter of 60 nm were prepared using sodium citrate as a reducing and stabilizing agent according to the literature. See, for example, Guerrero, A. R.; Aroca, R. F. Surface-Enhanced Fluorescence with Shell-Isolated Nanoparticles (SHINEF), Angewandte Chemie International Edition, 50, 665-668 (2011), which is incorporated herein by reference. Briefly, a total of 100 mL of 1 mM chloroauric acid aqueous solution was heated to boiling with vigorous stirring. After adding 4.2 mL of 1% trisodium citrate, the pale yellow solution turned fuchsia within several minutes. The colloids were refluxed for another 20 minutes to ensure complete reduction of Au ions, followed by cooling to room temperature.

### Example 2

This example details one embodiment of a method for making a diatomaceous earth plate for TLC by spin coating diatomaceous earth on glass slides. The diatomaceous earth was dried at 150° C. for 6 hours in an oven. After cooling to room temperature, 6 grams of diatomaceous earth was dispersed in 10 mL of a 0.5% aqueous solution of carboxymethyl cellulose and then deposited onto the glass slide for spin coating at 120 rpm for 20 seconds. The plates were placed in the shade to dry and then activated at 110° C. for 3 hours to improve the adhesion of diatomaceous earth to the glass substrate.

### Example 3

This example concerns a general TLC-SERS method. Disclosed embodiments of a TLC-SERS device and method for its use are particularly suitable for on-site detection of analytes from mixtures or biofluid. First, 1 μL of samples comprising a mixture of analytes, at least one of which is detectable by Raman spectroscopy, were spotted at 12 mm from the edge of a TLC plate. After drying in air, the TLC plate was kept in the glass TLC development chamber using a suitable mobile phase eluent. After analyte separation, the TLC Plates were then dried in an oven at 60° C. The separated analyte spots were marked under ultraviolet illumination at 380 nm and visualized by iodine colorimetry. The retention factors (Rf) of the analytes on TLC plates were calculated and marked on the TLC plates so that the analytes spots could be traced even when they are invisible at low concentrations. Then 2 μL of concentrated Au NPs were added directly to the analyte spots, and this nanoparticle addition step was repeated three times. A Horiba Jobin Yvon Lab Ram HR800 Raman microscope equipped with a CCD detector was used to acquire SERS spectra, and a 50× objective lens was used to focus the laser onto the SERS substrates. The excitation wavelength was 785 nm, and the laser spot size was 2 μm in diameter. The confocal pinhole was set to a diameter of 200 μm. SERS mapping images were recorded with a 10×10-point mapping array and were collected using a DuoScan module with a 2.0 μm step size, 0.5 s accumulation time, and collected in the Raman spectral range from 800 cm⁻¹ to 1800 cm⁻¹. The acquired data were processed with Horiba LabSpec 5 software.

### Example 4

This example generally concerns drug detection. In one working example, cocaine (C₁₇H₂₁NO₄) was chosen as the target analyte, which is an alkaloid derived from coca leaves. Cocaine is an illicit drug used widely all over the world. Instant on-chip testing of cocaine from biofluids, such as saliva, blood and urine, is very important in forensics and for medical diagnosis. A diatomaceous earth porous microchannel chip was fabricated and used for on-chip chromatography/SERS to separate and detect cocaine from real biofluid. The diatomaceous earth porous microchannel chip provided tight sample confinement of the target molecules, resulting in nearly 1000 times better level of detection compared to normal chromatography plates.

Cocaine was artificially added to plasma to obtain different concentrations (10 ppb to 100 ppm) and was applied to the diatomaceous earth porous microchannel chip. Cyclohexane and ethanol (v/v=6:1) were used as the eluent to separate cocaine from plasma. Certain additional bio-macromolecules, such as albumin, and tissue in plasma, do not diffuse on the porous microchannel due to the high molecular weight, whereas cocaine in the serum does migrate along the microchannel. Good separation and detection of cocaine therefore is achieved using a diatomaceous earth porous microchannel chromatography SERS method.

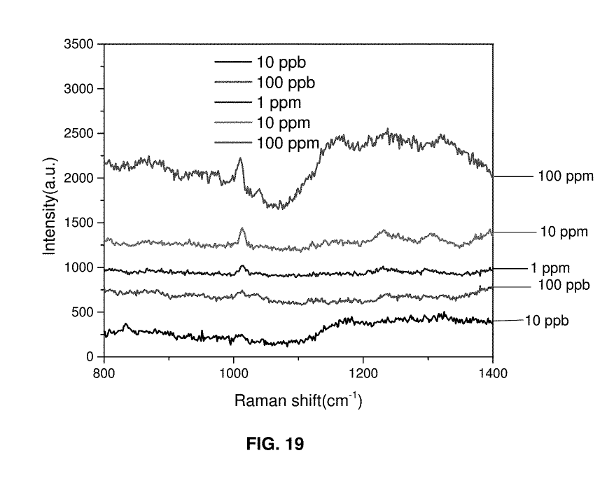

SERS spectra were obtained from for each of the samples having different concentration of cocaine in plasma, and these spectra are provided by FIG. 19. The Raman peak at 1008 cm⁻¹ was assigned to the aromatic ring breathing of cocaine. As shown by FIG. 19, the characteristic cocaine signal at 1008 cm⁻¹ exhibited a steady decrease in intensity as the cocaine concentration in plasma decreased. The detection limit for cocaine/plasma was 10 ppb on the diatomaceous earth porous microchannel, which is substantially better than liquid chromatography systems and is far below the 0.1-0.3 ppm level of cocaine that occurs in blood serum after cocaine use.

### Example 5

This example concerns polycyclic aromatic hydrocarbon (PAH) detection. Polychromatic hydrocarbons (PAHs) are a class of aromatic compounds comprising two or more aromatic or heterocyclic rings. PAHs are harmful for both public health and the environment. Unfortunately, the low binding affinity between PAHs and metallic substrates prevents efficient SERS detection of PAHs from mixtures as the spectra from co-existing components interfere with the spectrum from the PAHs. This example concerns using SERS to detect MBA, pyrene and mixtures thereof.

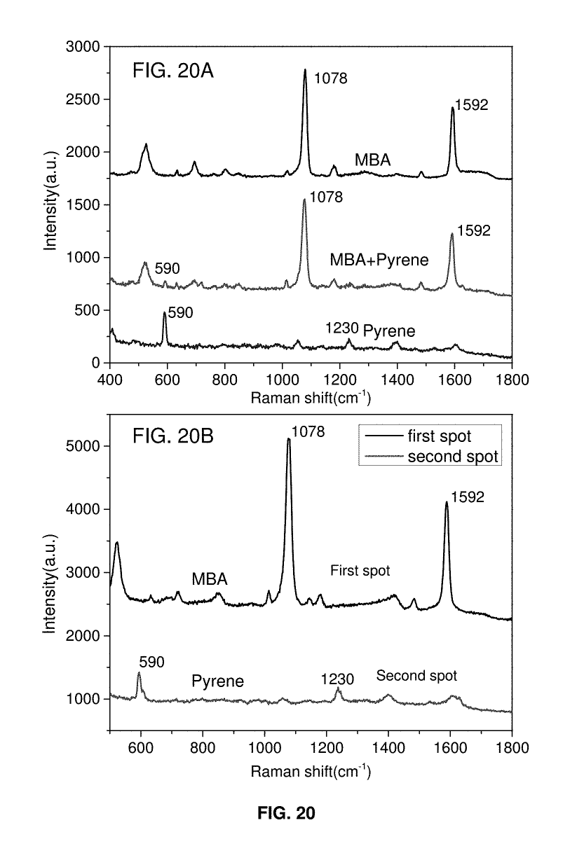

FIG. 20A provides the SERS spectra of MBA, pyrene and their mixture. MBA is a commonly used Raman probe molecule because of its affinity with metallic surfaces and intense Raman signals. The signals at 1074 cm⁻¹ and 1587 cm⁻¹ are associated with C—C ring-breathing modes of MBA. For the mixture (Pyrene and MBA 1/1), the metallic surface coverage was dominated by MBA because covalent bonds readily form between Au NPs and the MBA mercapto group. Thus only a very weak Raman peak from pyrene was observed from the SERS spectra of mixture. It is hard to distinguish the Raman peaks of the compounds in the mixture by normal SERS without separation technology.

When the fluid sample travels up the diatomaceous earth porous microchannel via capillary action, the diatomaceous earth functions as a stationary phase for chromatography. The numerous hydroxyl groups on the diatomaceous earth surface define a highly polar compound. After the mixture sample has been applied to the diatomaceous earth porous microchannel, a solvent fluid is drawn up the plate. More polar analytes have stronger interactions with the diatomaceous earth, and hence travel a shorter distance than less polar compounds in the mixture.

The separation effect of a diatomaceous earth porous microchannel with pyrene and MBA mixture was investigated. A hexane:ethyl acetate (v/v=6:1) was used as the eluent for the separation of pyrene from the mixture. After the fluid finished migrating on the diatomaceous earth porous microchannel, a UV lamp and iodine colorimetry were used to visualize different analyte spots corresponding to pyrene and MBA (See FIG. 17). The less polar pyrene traveled farther from the original application point because of its weak affinity with the polar diatomaceous earth. SERS spectra at corresponding spots were collected on the surface of diatomaceous earth porous microchannel as shown in FIG. 18B. The characteristic peaks of pyrene at 590 cm⁻¹ and 1230 cm⁻¹ are clearly observed. This example therefore established that the diatomaceous earth porous microchannel can successfully be used as the stationary phase in on-chip chromatography method.

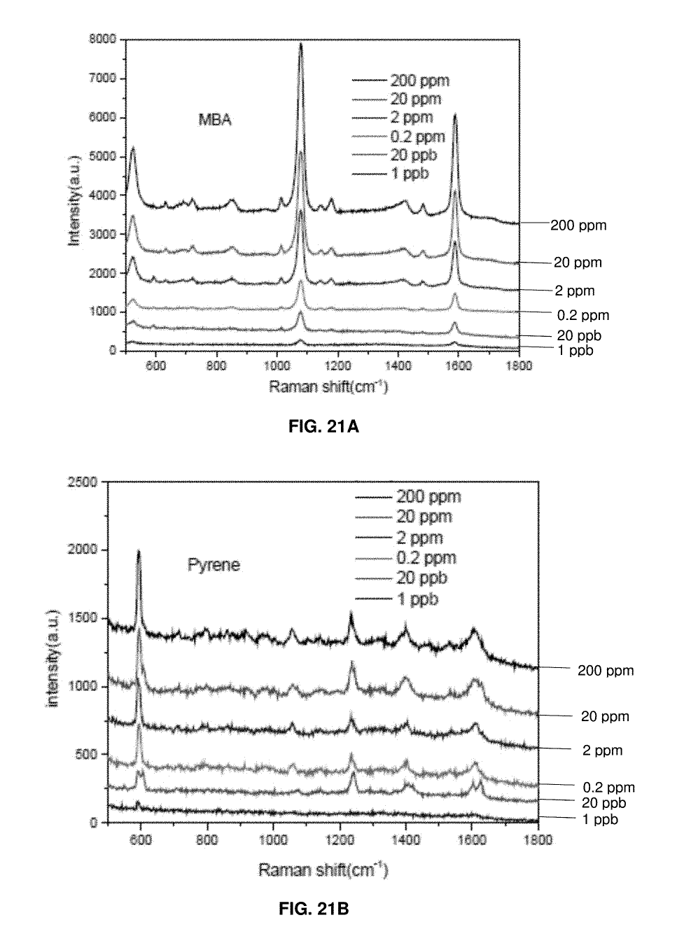

As shown by FIGS. 21A and 21B, all the characteristic bands of MBA and pyrene exhibit an incremental and steady decrease in intensity as the mixture concentration decreases. The detection limit from pyrene/MBA mixture is reduced to at least as low as 1 part per billion when using a porous diatomaceous earth microchannel chip according to the present invention. In contrast, the detection limit was only about 2 parts per million using the normal diatomaceous earth chromatography chip. Thus, these experimental results demonstrate more than three orders of magnitude (1000 times) sensitivity improvement that results by using the porous microchannel chip embodiments of the present invention compared to normal diatomaceous earth chromatography chip. Without being bound by a theory of operation, this substantial increase in sensitivity may be attributed to the relatively small dimensions of the microchannel, which reduces lateral diffusion of fluid flow during eluent migration, which leads to the target molecule being concentrated within the microchannel.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A method, comprising:
separating a composition comprising at least two separate components into a first component and a second component using diatomaceous earth as a stationary chromatography phase, wherein at least one of the first and second components is detectable by Raman spectroscopy; and
analyzing the separated components using Raman spectroscopy.

2. The method according to claim 1 wherein the composition includes at least one target molecule selected from an explosive, chemical warfare agent, drug, toxicant, pollutant, fire accelerant, gunshot residue, food adulterant, hazardous ingredient, or a combination thereof.

3. The method according to claim 2 wherein:
the explosive is selected from TNT, DNT, ammonium nitrate, TATP, PETN, RDX, TNB, DNAN, HMTD, or a combination thereof;
the chemical warfare agent is selected from sarin (GA), tabun (GB), VX, mustard gas (HD), 2-chloroethyl ethyl sulfide, triphenyl phosphate, dimethyl methyphosphonate, and a combination thereof;
the drug is selected from cocaine, heroin, morphine, codeine, nicotine, mefenorex, pentylenetetrazole, pemoline, caffeine, erythropoietin (EPO), hydrocodone, amphetamines, benzodiazepine species, or a combination thereof;
the pollutant is selected from carbendazim, imidacloprid, acetamiprid, phoxim, boscalid, buprofezin, myclobutanil, benzene, pyridine, xylene, formaldehyde, perchloroethylene, toluene, or a combination thereof;
the fire accelerant comprises a polycyclic aromatic hydrocarbon;
the gunshot residue comprises ethyl centralite, methyl centralite, or a combination thereof; and
the food adulterant and/or hazardous ingredient is selected from an antibiotic, dye, pesticide, hormone, contaminant, or a combination thereof.

4. The method according to claim 1, further comprising applying metal nanoparticles to the first and second components to increase signal intensity.

5. The method according to claim 4 where the metal nanoparticles are selected from gold (Au) nanoparticles (NPs), silver (Ag) nanoparticles, copper (Cu) nanoparticles, aluminum (Al) nanoparticles, or combinations thereof.

6. The method according to claim 5 wherein the nanoparticles have a diameter ranging from 10 nanometers to about 200 nanometers.

7. The method according to claim 1, wherein the diatomaceous earth is deposited on a substrate to provide a desired layer thickness.

8. The method according to claim 7 wherein the layer thickness is from greater than 0 μm to at least 100 μm.

9. The method according to claim 1, wherein the diatomaceous earth is provided as microchannel comprising or made from diatomaceous earth.

10. The method according to claim 9, wherein the microchannel is fluidly associated with an eluent reservoir.

11. The method according to claim 10, wherein the microchannel has a width of about 100 μm to about 1.0 millimeter, and the reservoir has a diameter of about 0.1 millimeter to 2 millimeters.

12. The method according to claim 1, comprising using a thin layer chromatography plate comprising a diatomaceous material layer having a thickness of from greater than zero μm to about 50 μm.

13. The method according to claim 12, wherein the thin layer chromatography plate comprises a diatomaceous material layer having a thickness of from about 10 μm to about 30 μm.

14. The method according to claim 12, providing a SERS intensity increase of at least 1 times up to at about 70 times relative to using a non-diatomaceous earth based stationary phase material.

15. The method according to claim 1 wherein the composition is a biological sample, and the method provides at least 10 times improved level of detection using a diatomaceous-earth-based TLC plate compared to using a commercially available silica-gel TLC plate.

16. The method according to claim 1 wherein the level of analyte detection is improved from about 2 times to at least about 10 times relative to using the same process with non-diatomaceous earth stationary phases.

17. The method according to claim 1 further comprising performing the separation and detection using a microchannel comprising or formed from diatomaceous earth.

18. A method, comprising:
providing a separation and detection device comprising a microchannel comprising or formed from diatomaceous earth
using the device to separate a composition comprising at least two separate components into a first component and a second component using diatomaceous earth as a stationary chromatography phase, wherein at least one of the first and second components is detectable by Raman spectroscopy, and wherein the composition includes at least one target molecule selected from explosives, chemical warfare agents, drugs, toxicants, pollutants, fire accelerants, gunshot residues, food adulterants, hazardous ingredients, and combinations thereof;
applying metal nanoparticles to the first and second components to increase signal intensity, wherein the metal nanoparticles are selected from gold (Au) nanoparticles (NPs), silver (Ag) nanoparticles, copper (Cu) nanoparticles, and aluminum (Al) nanoparticles; and
analyzing the separated components using Raman spectroscopy.

19. The method according to claim 29 wherein the nanoparticles are gold nanoparticles having a diameter of from about 50 nanometers to about 60 nanometers.

20. The method according to claim 29 where the device is a microfluidic diatomaceous earth analytical device comprising a microchannel having a width of about 100 μm to about 1.0 millimeter and being fluidly associated with an eluent reservoir having a diameter of about 0.1 millimeter to 2 millimeters.

21. A system, comprising:
a separation and SERS analysis device comprising at least one microchannel comprising or formed from diatomaceous earth; and
a Raman spectrometer.
